(19) United States
(12) Patent Application Publication
(10) Pub. No.: US 2024/0277585 A1
(43) Pub. Date: Aug. 22, 2024

(54) COMPOSITION OF LIPID NANOPARTICLE CONTAINING VITIS VINIFERA EXTRACT, COSMETIC USES OF A COMPOSITION OF LIPID NANOPARTICLE CONTAINING VITIS VINIFERA EXTRACT, ANTIOXIDANT DERMOCOSMETIC PRODUCT AND FOR PREVENTING SKIN AGING AND SKIN CARE METHOD

(71) Applicant: ACHÉ LABORATÓRIOS FARMACÊUTICOS S.A., Guarulhos (BR)

(72) Inventors: Cristiano Ruch WERNECK GUIMARAES, São Paulo (BR); Lisandra RAVANELLI PESSA, São Paulo (BR); Romulo DRAGANI REIS, Guarulhos (BR); Samuel VIDAL MUSSI, São Paulo (BR)

(21) Appl. No.: 18/681,117

(22) PCT Filed: Jul. 20, 2022
(86) PCT No.: PCT/BR2022/050269

§ 371 Date: Feb. 5, 2024

(30) Foreign Application Priority Data
Aug. 5, 2021 (BR) 102021015486-1

(51) Int. Cl. A61K 8/11; A61K 8/06; A61K 8/368; A61K 8/49; A61K 8/90; A61K 8/9789; A61Q 19/08

(52) U.S. Cl. CPC A61K 8/11; A61K 8/062; A61K 8/064; A61K 8/368; A61K 8/498; A61K 8/90; A61K 8/9789; A61Q 19/08; A61K 2800/413; A61K 2800/56

(57) ABSTRACT

The present invention aims to solve the constant problem of the state of the art related to the lack of stability of lipid nanoparticles containing Vitis vinifera extract, by elaborating a stable composition of lipid nanoparticles containing the Vitis vinifera extract and the use of nanoencapsulated Vitis vinifera L. extract as an active in cosmetic compositions, especially anti-aging compositions. Specifically, the present invention comprises less than 2.5% w/w of Vitis extract and an encapsulating system, which allows its better stabilization. The present invention is in the field of cosmetic preparations for treating the skin, especially with regard to liposome compositions involving a plant extract.

### FIELD OF THE INVENTION

The present invention relates to a composition of lipid nanoparticles comprising Vitis vinifera extract, nanoparticles, cosmetic composition comprising said nanoparticle and cosmetic uses thereof. The present invention is in the field of cosmetic preparations for skin treatment, especially referring to solid composition of lipid nanoparticles involving a plant extract.

### BACKGROUND OF THE INVENTION

The skin is the largest and most visible organ of the human body and stands out for playing fundamental roles in maintaining life. Among the multiple functions performed, they include protection against various mechanical injuries, invasion of parasites and microorganisms, chemical agents and radiation. Under radiation, the skin exerts a daily protection against sunlight (UV).

Another key role is protection against fluid loss and body temperature regulation.

As a result of its physiological functions, the skin is daily damaged by exogenous factors, which favor the appearance of some unwanted conditions such as, for example, pigmentation disorders, wrinkles, dehydration, among other aggressions to the skin. These undesirable conditions are further favored by different endogenous factors, which are triggered mainly with advancing age.

Thus, there is a continuing need to develop cosmetic compositions that favorably alter and help to treat, inhibit or prevent such undesirable skin conditions.

Currently, there is a wide variety of active ingredients, chemical or biological, proposed for the treatment of a variety of skin disorders. These actives are routinely added in cosmetic bases for topical use. On the market, the most common cosmetic forms are creams, gel-creams, gels, ointments, serums and lotions, which can vary from aqueous compositions to water-free compositions, which include several types of emulsions (oil-in-water or water-in-oil).

The active ingredients chosen for use in cosmetic forms tend to have antiinflammatory, photoprotective (UVA and UVB), antioxidant, moisturizing, whitening and anti-wrinkle properties; some promote skin elasticity increase, others improve skin touch sensation. It is always desirable that cosmetic actives have physical and chemical stability. It is also desirable that they have satisfactory skin absorption and/or that, when formulated in cosmetic bases, they have improved stability, bioavailability and skin permeation.

Regarding the biological active ingredients, the grape extract (belonging to the genus Vitis, species Vitis vinifera) is an active recognized for its ability to act favorably in the cosmetic treatment of undesirable skin conditions. This ability of Vitis vinifera extract is associated with a large amount of phenolic compounds, among which anthocyanins stand out, in addition to flavonoids, procyanidins, catechins and phenolic acids, such as gallic acid. Additionally, the Vitis vinifera extract also includes the presence of glycosides from the flavonoids quercetin, kaempferol, myricetin and isoramnetin, as well as coumaric, caffeic, ferulic and caftaric acids, in addition to resveratrol. Malvidin 3-O-glycoside is also a compound present in the extract.

Thus, said extract has several protective and cosmetic effects against various conditions such as pigmentation disorders, wrinkles, dehydration, chloasma, acne vulgaris, redness, among others.

However, the use of Vitis vinifera extract in cosmetic bases is limited by a constant technical problem related to its low stability, as it is an extract sensitive to different environmental conditions (light, oxygen, temperature). Low stability is inherently related to the extract's ability to perform biological functions beneficial to the skin. For example, resveratrol, an active ingredient commonly present in Vitis vinifera extract, is a highly photosensitive molecule that, when exposed to light, converts from the trans-(active) isomer to the cis- (inactive) isomer.

Normally, instability of Vitis vinifera extract is observed, either alone or formulated into a cosmetic base. Instability is observable by the change in organoleptic (especially color) and physicochemical (pH and density) properties.

In the search for the state of the art in scientific and patent literature, the following documents related to the subject of the present invention were found:

WO2011116963, published in 2011, teaches that solid lipid particles (SLN—Solid lipid nanoparticles) exhibit improved chemical stability properties of the encapsulated species when the SLN is coated with a polymer. The stability described in the international application is related to greater protection of the encapsulated species against degradation by interaction with other ingredients of the composition, as well as other chemical protections, such as against hydrolysis, oxidation and light.

In the application WO2011116963 a series of actives to be encapsulated is presented, being the Vitis vinifera extract just one of several suggested plant extracts.

Thus, from the researched literature, the lipid nanoparticles of Vitis vinifera extract described in the state of the art still present the problem of lack of stability of the extract, as the lack of stability lies both in a composition of lipid nanoparticles containing Vitis vinifera alone, or when formulated in a cosmetic base. In the state of the art, there is also no effective solution to improve skin penetration. Therefore, there is still a need to provide compositions of lipid nanoparticles, whose ingredients solve the lack of stability when encapsulating the Vitis vinifera extract, particularly with improvement in skin permeation, among other benefits, so that it becomes viable for its cosmetic purposes.

### SUMMARY OF THE INVENTION

The present invention aims to solve the constant problem of the state of the art related to the lack of stability of composition of lipid nanoparticles containing Vitis vinifera extract, through the elaboration of a composition of improved stability, which enables the use of nanoencapsulated Vitis vinifera L. extract as an active in cosmetic compositions, especially anti-aging compositions.

In a first aspect, the present invention relates to a composition of lipid nanoparticles containing Vitis vinifera extract in an amount of about 0.1% to 2.0% by weight of extract with respect to the total weight of the composition, more specifically about 1% by weight of extract with respect to the total weight of the composition.

In a second aspect, the present invention relates to cosmetic uses of the composition of lipid nanoparticles of the first aspect in preventing skin aging, as an antioxidant and anti-aging. Surprisingly, the composition of the present invention proved to be efficient in improving skin permeation, as well as promoting a reduction in the oxidative stress of the skin, exerting a protective effect against the excessive increase in the synthesis of free radicals, induced by exposure to UV radiation.

In a third aspect, the present invention relates to a dermocosmetic product for preventing skin aging, acting as an antioxidant and anti-aging, comprising at least a composition of lipid nanoparticles of the first aspect and cosmetically acceptable excipients.

In a fourth aspect, the present invention relates to a method of skin care, comprising a step of topically applying a layer of a product as defined in the third aspect.

Furthermore, the inventive concept common to the described and claimed protection contexts is that they all refer to a stable composition of Vitis vinifera extract lipid nanoparticles, which is surprisingly and unexpectedly improved in relation to the state of the art by (i) remaining in a homogeneous system, (ii) with its organoleptic properties preserved even under stress conditions, (iii) presenting a high encapsulation index, (iv) improving skin penetration, (v) exerting an anti-aging effect and (vi) exerting an antioxidant effect, protecting the skin from the deleterious effects of exposure to solar radiation.

These and other objects of the invention will be immediately appreciated by those skilled in the art and will be described in sufficient detail for their reproduction in the following description.

### BRIEF DESCRIPTION OF THE FIGURES

In order to better define and clarify the content of the present patent application, the following figures are presented:

FIG. 1 shows a comparison of the stability profile between three compositions of Vitis vinifera extract lipid nanoparticles substantially identical when subjected to the stability test in an oven (52° C. for 24 hours). Composition A comprises 1% extract, composition A′ comprises 1.5% and composition A″ comprises 2%.

FIG. 2 shows three compositions of Vitis vinifera extract lipid nanoparticle substantially identical containing 2.5%, 5.0% and 10.0% extract. The figure demonstrates the infeasibility of the comparative compositions, due to the formation of clusters and high viscosity, preventing the obtaining of nanoparticles.

FIG. 3 presents a calibration curve for total polyphenols, showing an encapsulation efficiency of 99.97%.

FIG. 4 shows fluorescent micrograph evaluation of skin permeation in culture of human skin fragments incubated with the evaluated products. A-C—Untreated skin fragments (Baseline Control); D-F—Skin fragments incubated with the evaluated product NVAC VITIS VINI (BLANK)+FLUORESCEIN; G-I—0.1% EXTRACT SOLUTION+FLUORESCEIN; J-L—NVAC VITIS VINI 10%. M-O—NVAC VITIS VINI 10%+FLUORESCEIN. Fluorescein is marked in red. The reference bar corresponds to 20 μm.

FIG. 5 shows evaluation of cutaneous permeation in culture of human skin fragments with the evaluated products NVAC VITIS VINI 10%, NVAC VITIS VINI 10%+FLUORESCEIN, NVAC VITIS VINI (BLANK)+FLUORESCEIN and EXTRACT SOLUTION 0.1%+FLUORESCEIN. Data represent the mean±standard deviation of 12 replicates (ANOVA—Bonferroni).

FIG. 6 shows a fluorescent micrograph evaluation of the synthesis of free radicals (FRs) in culture of human skin fragments incubated with the evaluated products and exposed to ultraviolet (UV) radiation. Where: A-C—Untreated skin fragments; (Baseline Control); D-F—Skin fragments only exposed to UV radiation; G-I—Skin fragments incubated with the evaluated product NVAC VITIS VINI (BLANK)+FLUORESCEIN and subjected to UV radiation; J-L—Skin fragments incubated with the evaluated product EXTRACT SOLUTION 0.1%+FLUORESCEIN and subjected to UV radiation. M-O—Skin fragments incubated with the evaluated product NVAC VITIS VINI 10% and subjected to UV radiation. P-R—Skin fragments incubated with the evaluated product NVAC VITIS VINI 10%+FLUORESCEIN and subjected to UV radiation. S-U—Skin fragments incubated with the market product for comparison, VIT C 10% MOISTURIZING CREAM and subjected to UV radiation. The FRs are marked in green mainly on the dermis and the blue marking represents the cell nucleus (DNA; DAPI). The reference bar corresponds to 50 μm.

FIG. 7 presents the results of the semi-quantification of labeling/production of FRs obtained from the analysis of microscopic images.

FIGS. 8 and 9 show the size and surface charge of the nanoparticles according to the present invention.

### DETAILED DESCRIPTION OF THE INVENTION

The present invention relates to a stable composition of lipid nanoparticles containing Vitis vinifera extract and the use of the composition containing nanoencapsulated Vitis vinifera L. as active in dermocosmetic compositions, especially antioxidant and anti-aging compositions.

For purposes of the present invention, unless otherwise stated, percentage values refer to weight/weight percentage (% w/w). Thus, percentages mean the weight of one or more ingredients in relation to the total weight of the composition.

In the invention, the term “lipid nanoparticles” can be understood as systems for releasing an active in a spherical shape, with a diameter from 1 to 1000 nanometers, whose matrix is formed by lipids. Lipid nanoparticles are a system that encapsulates at least the Vitis vinifera extract. Furthermore, in the present invention the nanoparticles containing the Vitis vinifera extract is an input to cosmetic compositions, especially for having cosmetically active properties.

In the present invention the term “Vitis vinifera extract”, can also be understood as “Vitis extract” or “grape extract”, and is defined as an extract of part (peel or seed) or all of the grape. Vitis extract can be obtained through any method known to the state of the art of extracting grape components, being preferred those that maintain the quality and quantity of the ingredients so that the extract can perform its cosmetic functions.

In a particular embodiment, the Vitis vinifera extract according to the present invention comprises gallic acid, catechin, epicatechin, procyanidin B1 and procyanidin B2.

In a non-exhaustive indication, the process of obtaining the extract may involve water as a solvent or a mixture of water with other solvents, for example, alcohol. The process may involve different unit operations for the industrial transformation of part (seed or skin) or all of the grape into an extract, such as: grinding, solvent extraction, filtration, concentration, drying, sieving and packaging.

“Extract” means fractions obtained from Vitis vinifera, which can be enriched with specific compounds, for example phenolic compounds, becoming fractions not naturally achievable without human intervention.

The composition of the present invention is for the preparation of lipid nanoparticles containing Vitis vinifera extract, which stands out for having an improved stability effect when compared to other lipid nanoparticles containing the same extract. The composition stability of the present invention is given by its ability to maintain its homogeneity, its organoleptic and physicochemical properties, even when subjected to stress conditions foreseen in stability tests.

The stability of the nanoparticle composition of the present invention is directly related to its quality and chemical and physical integrity, as well as its useful life and feasibility in being formulated in other cosmetic bases.

The composition of lipid nanoparticles of the present invention is composed of a lipid phase, also called lipid matrix, which enables the encapsulation/retention of ingredients. The lipid phase is formed by at least one lipid, selected from a range of compounds such as, non-exhaustively, mono-, di- and triglycerides, fatty acids, sterols and waxes. The lipid phase may contain only solid lipids, only liquid lipids, or a mixture of solids and liquids. The lipid phase may be stabilized by the presence of at least one surfactant and may have a polymeric coating.

The nanoparticles of the present invention can be produced by different processes known in the state of the art, such as, for example, ultra-homogenization and high pressure homogenization, which can be hot or cold, as well as the solvent evaporation method, multiple emulsions or spontaneous emulsification. Preferably, the nanoparticles are produced by the high pressure homogenization process.

If a polymeric coating is desired, an additional coating step takes place, by deposition or coupling of the polymer(s), which can be isolated or mixed on the surface. This deposition can be done using low-energy methods, such as simple homogenization with a magnetic mixer and paddles, or high-energy methods, such as high-pressure homogenizers, ultrasound or ultra-homogenizers. Moreover, it can be performed during the process of obtaining the nanoparticle, immediately or a long time after obtaining the lipid nanoparticles still uncoated. The coupling of polymers can be achieved through chemical reactions on the surface of the nanoparticle, by simple and short mixing or by extended incubation.

The lipid nanoparticles of the present invention encapsulate ingredients within the lipid matrix. The encapsulated ingredients are Vitis extract and cosmetic adjuvants. By adjuvant is meant any ingredient that can provide additional beneficial effects onto the skin or on the composition itself.

Adjuvants are substances that may or may not have a cosmetic effect. When it has a cosmetic effect, adjuvants can be emollients, humectants, antioxidants, chemical or physical filters for sun protection, and are not limited to just these functions.

In another case, adjuvants are substances that have a stability effect, for example, preservatives, antioxidants, chelators.

Surfactants and/or solvents can be used to solubilize and stabilize the encapsulated ingredient within the lipid matrix, as well as make it possible to obtain nanoparticles by stabilizing the interface of the lipids with the aqueous phase.

The substances encapsulated by the lipid nanoparticle of the present invention can be lipophilic, hydrophilic or amphiphilic and can be incorporated into the lipid matrix by solution or dispersion in the lipid, by adsorption on the surface of the lipid or by dispersing the active ingredient in the lipid in the form of an aqueous solution, depending on the chosen preparation process.

In the present invention, the lipid nanoparticles are found in the form of an aqueous dispersion. The nanoparticle composition has at least 50% by weight in relation to the total weight of the water composition.

The significant amount of water present in the composition can favor the formation of a condensate in the formulated product, especially when it is subjected to high temperatures. On the other hand, such a condensate is foreseen and does not negatively influence the stability of the composition.

In a first aspect, the present invention relates to a composition of lipid nanoparticles containing Vitis vinifera extract in an amount of about 0.1 to 2.0% w/w by weight of extract in relation to the total weight of the composition.

Preferably, the amount of Vitis extract in the composition is between about 0.5 to 1.5% w/w. More preferably, the amount of Vitis extract in the composition is about 1.0% w/w.

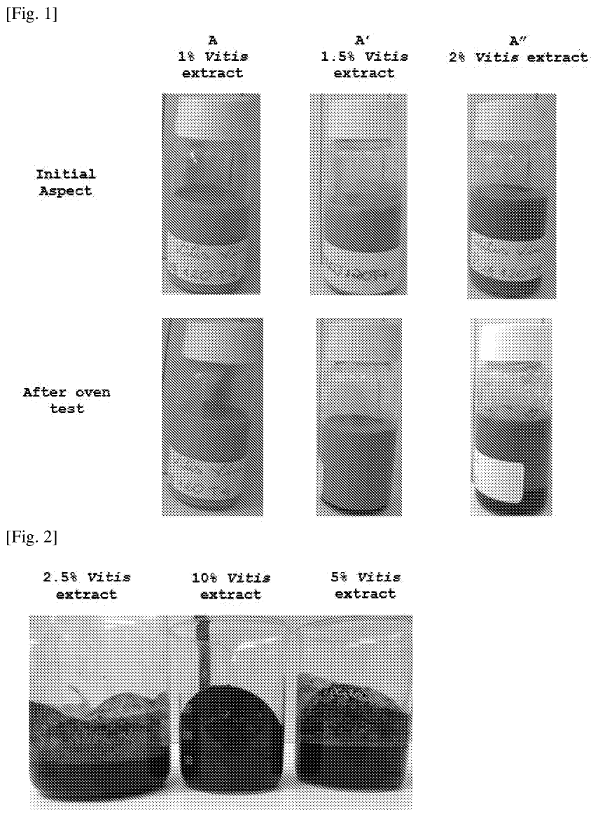

Less than 2% w/w of Vitis extract is technically relevant, as compositions of lipid nanoparticles containing amounts equal to or greater than about 2.0%, when subjected to the stability test in an oven (52° C./24 h), are shown to be unfeasible, as shown in [FIG. 1]. Higher amounts of Vitis extract in the composition culminate in the formation of clusters, occurrence of phase separation, technical problems that imply in obtaining a heterogeneous system, which characterizes lack of stability of the prepared composition. At levels of 5% and 10%, for example, the composition becomes unfeasible, forming a paste that makes solubilization and encapsulation impossible ([FIG. 2]).

The amount of Vitis extract of the present invention differs from the prior art knowledge, which does not teach or suggest a specific amount of Vitis extract and which could consider that any encapsulated amount would benefit from a stability effect, which is not observable in industrial practice ([FIG. 1] and [FIG. 2]).

Surprisingly, the extract of the present invention, formulated in an amount of about 0.1 to 2.0% by weight, remained as a homogeneous system, even when exposed to a temperature of 52° C. for 24 h, demonstrating its good physical stability, such an effect was intensified in amounts close to 1.0%. Additionally, in amounts from about 0.1 to about 2.0%, a high encapsulation index is observed.

Another surprising aspect observed in the present invention is the softness of the color obtained, ranging from soft purple to pink.

In another embodiment of the first aspect, the invention is formulated with about 0.1 to about 3% of a preservative. Suitable preservatives for the present invention are: phenoxyethanol, caprylyl glycol, BHT, disodium EDTA, sodium metabisulfite, parabens, Lonicera japonica, Lonicera caprifolium, hydroxyacetophenone, 1,2-hexanediol, 1,2-octanediol, tropolone, pentylene glycol, sodium benzoate, potassium sorbate, iodopropynyl-butylcarbamate, imidazolidinyl urea, polyaminopropyl biguanide or a mixture thereof. Preferably, the composition of the first aspect comprises at least 0.1 to 2% imidazolidinyl urea, 0.1 to 2% phenoxyethanol, 0.1 and 2% caprylyl glycol or a combination thereof.

The choice of preservative system is relevant to the present invention as it impacts on the improvement of the stability of the formulated product. The preservative prevents changes in organoleptic properties, such as coloring, and physicochemical properties of the composition. In addition, it inhibits microbiological growth. It is a challenge in the state of the art to provide an efficient preservative system that is compatible with the Vitis extract.

In an embodiment of this aspect, the preservative system used acts to maintain the stability of the composition, and microbiological control, and it comprises at least imidazolidinyl urea or a near 1:1 combination of phenoxyethanol and caprylyl glycol, both at a concentration of about 1.5% w/w. The efficiency of these preservative systems of the present invention is noted, for example, through a stability test and microbiological control (Challenge test). Even more preferably, the preservative system used in the present invention involves a combination of phenoxyethanol and caprylyl glycol, both at a concentration of about 1.5% w/w. The preservative system added to an amount of Vitis extract of less than about 2.0% in the form of lipid nanoparticles confers stability to the Vitis extract and the nanoparticle of the present invention.

The composition of the present invention comprises about 10% to 40% w/w lipid for forming the lipid matrix. At room temperature, the lipids used to form the matrix can be lipids in liquid, semi-liquid, solid state or a mixture thereof. On a non-exhaustive basis, lipids are selected from simple or complex fatty acids, long, medium or short fatty chain triglycerides.

Preferably, the present invention uses about 10% to 30% medium chain triglycerides, fatty acids and polypropylene glycol stearyl ester. More preferably, the present invention uses 5% to 10% capric/caprylic acid triglyceride, 5% to 10% oleic acid, 1% to 10% linoleic acid and 1% to 5% PPG-15 stearyl ether.

The composition of the first aspect comprises about 1% to 10% surfactants. Surfactants can be selected from hydrophilic and lipophilic, ionic and non-ionic compounds or a mixture thereof.

Preferably, the composition uses 1 to 10% non-ionic surfactants selected from ethoxylated surfactants such as steareth (stearyl alcohol ethoxylate) and poloxamer 407 (polymer with oxirane). More preferably, the composition uses 1 to 5% steareth-2 (stearyl alcohol ethoxylate), 1 to 5% poloxamer 407 (polymer with oxirane) and 0.1 to 2% steareth-21 (stearyl alcohol ethoxylate).

The first aspect of the present invention relates to a composition comprising at least the following components: more than 50% water, about 10 to 40% lipids, about 1 to 10% surfactants, about 0.1 to 3% adjuvants and about 0.1 to 2% Vitis extract, preferably 1% Vitis extract.

In the present invention, the composition may optionally further comprise polymers for coating the nanoparticles. The composition may comprise 0.5% to 5% hydrophobic and hydrophilic polymers. The polymers can be selected from acrylic acid-derived polymers, polylactic acid-derived polymers, polymethacrylates, ethylene and vinyl acetate copolymer, vinylpyrrolidone-derived polymers, ethylene oxide and propylene oxide non-ionic block copolymer, cellulose hydroxypropylmethyl ether polymer, hydroxyethyl cellulose, hydroxypropyl cellulose, ethyl cellulose, cellulose acetate phthalate (phthalic anhydride polymer and cellulose acetate ester) carboxymethyl cellulose, cellulose acetate (cellulose polymer partially acetylated to different degrees).

In a second aspect, the present invention relates to the cosmetic use of the composition of lipid nanoparticles of the first aspect to prevent skin aging by exerting antioxidant and anti-aging effects.

The cosmetic use of the second aspect of the present invention derives from the fact that the composition of lipid nanoparticles containing Vitis extract has antioxidant, anti-aging, anti-inflammatory, whitening effect, photoprotection, gene modulation activities.

The effects are achieved by the improved performance on skin permeation.

The antioxidant effect of the composition of the first aspect was determined by the methods of DPPH (2,2-diphenyl-1-picrylhydrazyl), SOD effect (Superoxide Dismutase) and DCFH-Da (2,7-Dichlorodihydrofluorescein-diacetate). By using the respective methods, it was observed that the composition had: (i) an antioxidant capacity 4× superior to Resveratrol, (ii) 12× superior to Vitamin C and (iii) 24× superior to Vitamin E; (iv) showed SOD effect on free radical scavenging; (v) showed the ability to scavenge free radicals in the intracellular environment.

The anti-aging effect of the composition of the first aspect was determined by the methods that measure the activity of the elastase enzyme and the activity of the metalloproteases (MMP) enzyme. By using the respective methods, it was observed that the composition has: (i) ability to inhibit the activity of elastase—Vitamins C and E do not present such result; (ii) ability to inhibit the activity of 3 different types of MMP (1, 3 and 12)—Vitamins C and E do not present this result.

The anti-inflammatory effect of the composition of the first aspect was determined by evaluating the activity of the cyclooxygenase 2 (COX-2) enzyme. By using said method, it was observed that the composition inhibited the expression of the COX-2 enzyme.

The whitening effect of the composition of the first aspect was determined by evaluating the activity of the tyrosinase enzyme. By using the method, it was observed that the composition reduced the activity of the tyrosinase enzyme, presenting results comparable to market references, such as kojic acid and arbutin.

The photoprotection effect of the composition of the first aspect was determined by microscopic evaluation of the integrity of reconstituted human skin, submitted to UV radiation. By using the method, it was observed that the composition is able to protect cells against damage caused by UVA and UVB radiation.

The gene modulating effect of the composition of the first aspect was determined through a panel containing several genes. More specifically, the effect of gene modulation was evaluated through mRNA expression profiling using RT-qPCR technology. The extracted mRNA was analyzed using PCR designed to analyze target genes selected for their importance in skin biology. By using the method, it was observed that the composition of the present invention is able to modulate genes related to skin aging.

The skin permeation-enhancing effect and antioxidant effect were also demonstrated by pre-clinical evaluation of skin permeation and antioxidant effect of the products evaluated in an ex vivo human skin experimental model.

In a third aspect, the present invention relates to a product for preventing skin aging, contemplating antioxidant and anti-aging effects, comprising the composition of lipid nanoparticles of the first aspect and cosmetically acceptable excipients.

The third aspect product may comprise ingredients that have cosmetically active function or not. Among the ingredients with a cosmetic function, it is highlighted any ingredients formulated for the preparation of a dermatological product such as, in nonexhaustive examples, moisturizers, skin lighteners, emollients, anti-wrinkles, antiimperfections, which increase the firmness and elasticity of the skin and that give a velvety touch to the skin. Among the ingredients with no cosmetic function, any ingredients that aim to make the desired cosmetic form possible, be it liquid, semi-solid or solid, stand out.

When in liquid form, the product of the present invention can be prepared as lotions and serums.

When in semi-solid or solid form, the product ofthe present invention can be prepared as oil-in-water or water-in-oil emulsions, preferably in cream or gel-cream form.

Furthermore, the product may be in gel preparation.

In a fourth aspect, the present invention relates to a method of skin care, including a step of applying to the skin a layer of a product as defined in the fourth aspect.

### EXAMPLES—EMBODIMENTS

The examples shown herein are intended to illustrate some of the numerous ways to carry out the invention, however without limiting its scope, with the purpose of helping to prove the technical effect, in a direct and comparative way.

The direct way of proving the technical effect will be through the demonstration that composition A (invention) is stable in different stability tests, in particular the tests that subject the composition to stress conditions. The indirect way will be through the comparative compositions from B to J, which even with similarities to the base composition, due to differences in the amount of Vitis extract and/or the preservative system, did not pass the stability tests under stress conditions.

### Example 1—Composition of Lipid Nanoparticles a (Invention)

TABLE 1
Composition of lipid nanoparticles A (Invention)

| Ingredient | Concentration (% w/w) |
|---|---|
| Water | 69.87% |
| Capric/Caprylic Acid | 7.0% |
| Triglyceride | |
| Oleic acid | 7.0% |
| Linoleic acid | 5.0% |
| PPG-15 stearyl ether | 4.0% |
| Poloxamer 407 | 1.5% |
| Steareth-2 | 2.0% |
| Steareth-21 | 1.0% |
| Vitis vinifera extract | 1.0% |
| Phenoxyethanol | 0.8325% |
| Caprylyl glycol | 0.6675% |
| BHT | 0.05% |
| disodium EDTA | 0.05% |
| Sodium Metabisulfite | 0.03% |

TABLE 2
Defined quality control parameters for the compositions of lipid
nanoparticles formulated with Vitis vinifera extract

| Parameter | Specification |
|---|---|
| pH | 2.5-4.5 |
| Density (g/mL) | 0.9-1.1 |
| Aspect | Milky liquid |
| Dispensability | Dispersion of encapsulated actives in water |
| Color | Purple to pink |
| Odor | Characteristic |

### Example 2—Stability Comparison of Composition a, with 1% Vitis vinifera Extract, to Compositions A′ (1.5%) and A″ (2%)

Compositions A, A′ and A″ shown in [FIG. 1] refer to three different compositions, identical in qualitative aspects and significantly similar in quantitative aspects, with only small adjustments in the amount of Vitis extract used in each of them. Composition A is as shown in Table 1, comprising 1% of Vitis extract, A′ comprises 1.5% and composition A″ comprises 2.0%.

The previous stability of each prototype was studied in an oven test (condition of 54° C. for 24 hours). Tables 3 and 4 present the variations observed in the appearance of the three samples.

TABLE 3
Organoleptic variations in the stability study of the compositions
of lipid nanoparticles A, A′ and A″ in the oven test at 54° C./24 h.

| Observed | Composition | Composition | Composition |
|---|---|---|---|
| Parameter | A (1%) | A′ (1.5%) | A″ (2%) |
| Viscosity | − | − | − |
| Change | | | |
| Curdling | − | − | − |
| Odor change | − | − | − |
| Color change | − | − | − |
| Precipitate | − | + | + |
| Presence of | − | − | + |
| clusters | | | |
| Presence of | + | + | + |
| condensation water | | | |
| Presence of | − | − | − |
| oil on the surface | | | |
| Suspension | − | + | + |
| separation | | | |

TABLE 4
pH variations in the stability study of the compositions
of lipid nanoparticles A, A′ and A″
in the oven test at 54° C./24 h.

| Observed | Composition | Composition | Composition |
|---|---|---|---|
| Parameter | A (1%) | A′ (1.5%) | A″ (2%) |
| Initial pH | 4.04 | 3.72 | 3.62 |
| Final pH | 4.17 | 3.49 | 3.66 |

In conclusion, it was observed that composition A (Table 1) is the most promising, mainly because it does not present precipitate after completion of the stability study in an oven at 54° C./24 h. The precipitate observed in the samples of compositions A′ and A″ is purple in color and is characteristic of a higher concentration of Vitis vinifera extract in the composition than the system supports. The pH, however, did not change significantly during the study; the pH variation was from 4.04 to 4.17 for sample A, from 3.72 to 3.49 for sample A′ and from 3.62 to 3.66 for sample A″.

[FIG. 1] illustrates the aspects of the three compositions (A, A′ and A″) before and after the test in an oven at 54° C./24 h.

### Example 3—Additional Stability Studies of the Composition of Lipid Nanoparticles A

After the previous stability study in an oven at 54° C./24 h, a set of additional tests was carried out to further investigate the stability of the composition of the composition of lipid nanoparticles A. The tests are in accordance with good practices for cosmetic stability studies and they consider the parameters defined in Table 2 as indicative of stability.

### Test 1: Centrifugation at 6000 Rpm/30′

The stability of the composition of lipid nanoparticles A of the present invention was evaluated by the centrifugation test at 6000 rpm/30′, herein referred to as Test 1. The methodology used involved the conditioning of a sample of the nanoparticles in eppendorf, followed by the exposure of the container to a rotation at a speed of 6000 rpm for 30 minutes at 25° C. At the end of the test, the integrity of the sample was evaluated in relation to suspension separation or phase separation.

After performing the test, no phase separation was observed in the composition, which remained homogeneous.

Therefore, the sample containing the composition of lipid nanoparticles A was considered stable in view of the simulation of a stress situation that can lead to phase separation.

### Test 2: Oven 54° C./24 h.

The stability of the composition of lipid nanoparticles A of the present invention was evaluated again by the 54° C./24 h oven test, herein referred to as Test 2. The methodology used involved exposing a sample of composition A to a temperature of 54° C. for 24 hours and at the end, the sample was evaluated for organoleptic characteristics and physicochemical aspects. As mentioned above, the criteria considered in the evaluation were: suspension separation, presence of oil on the surface, presence of condensation water, presence of clusters, presence of precipitate, curdling, color change, odor change, viscosity change. As for the physical-chemical parameters, in addition to the pH variation, the density variation was also evaluated.

The results of Test 2 for the sample of the composition of lipid nanoparticles A are shown in Tables 5 and 6.

TABLE 5
Organoleptic variations in the study of the stability of the composition
of lipid nanoparticles A in the oven test at 54° C./24 h.

| Organoleptic parameters of sample A | Result |
|---|---|
| No changes | − |
| Viscosity Change | − |
| Curdling | − |
| Odor change | − |
| Color change | − |
| Precipitate | − |
| Presence of clusters | − |
| Presence of condensation water | + |
| Presence of oil on the surface | − |
| Suspension separation | − |

TABLE 6
Variation of pH and density in the stability study of the composition
of lipid nanoparticles A in the oven test at 54° C./24 h.

| | Before Test 2 | After Test 2 | Expected |
|---|---|---|---|
| Parameter | (Sample A) | (Sample A) | Range |
| pH | 4.04 | 4.17 | 2.5-4.5 |
| Density (g/mL) | 0.99 | 0.99 | 0.9-1.1 |

Tables 5 and 6 allow us to conclude that the organoleptic and physicochemical parameters evaluated are within the expected ranges.

### Test 3: Preliminary Stability Test: Thermal Shock

The stability of the composition of lipid nanoparticles A of the present invention was evaluated by the test of sudden changes in temperature (thermal shock), herein called Test 3. The methodology used involved submitting the sample to 7 cycles of thermal stress, each cycle corresponding to a time of 24 h at 5° C., followed by 24 h at 40° C. At the end of each cycle, the organoleptic characteristics and physicochemical aspects were evaluated. The criteria considered in the evaluation were again: suspension separation, presence of oil on the surface, presence of condensation water, presence of clusters, presence of precipitate, color change, odor change, viscosity change, pH and density.

The results of Test 3 for the sample of the composition of lipid nanoparticles A are shown in tables 7, 8 and 9.

TABLE 7
Density variation of composition of lipid nanoparticles A before and
after completion of heat shock test - total of 7 heat shock cycles.

| | Before Test 3 | After Test 3 | Expected |
|---|---|---|---|
| Parameter | (Sample A) | (Sample A) | Range |
| Density (g/mL) | 0.99 | 0.99 | 0.9-1.1 |

TABLE 8
pH variation of the composition of lipid
nanoparticles A over 7 heat shock cycles.

| | pH of sample A |
|---|---|
| Test Stage 3 | (expected range 2.5-4.5) |
| Before the test (C0) | 4.04 |
| 1^{st} thermal shock (C1) | 3.61 |
| 2^{nd} thermal shock (C2) | 3.41 |
| 3^{rd} thermal shock (C3) | 3.32 |
| 4^{th} thermal shock (C4) | 3.25 |
| 5^{th} thermal shock (C5) | 3.29 |
| 6^{th} thermal shock (C6) | 3.39 |
| 7^{th} thermal shock (C7) - | 3.28 |
| Final | |

TABLE 9
Organoleptic variations observed in the composition
of lipid nanoparticles A over 7 heat shock cycles.

| Organoleptic | | | | | | | |
|---|---|---|---|---|---|---|---|
| parameters of | | | | | | | |
| sample A | C1 | C2 | C3 | C4 | C5 | C6 | C7 |
| Suspension separation | − | − | − | − | − | − | − |
| Presence of oil | − | − | − | − | − | − | − |
| on the surface | | | | | | | |
| Presence of clusters | − | − | − | − | − | − | − |
| Precipitate | − | − | − | − | − | − | − |
| Color change | − | − | − | − | − | − | − |
| Odor change | − | − | − | − | − | − | − |
| Viscosity change | − | + | + | + | + | + | + |
| Presence of | + | + | + | + | + | + | + |
| condensation water | | | | | | | |

In Tables 8 and 9, C1 to C7 refer to each thermal shock cycle in test 3.

Tables 7 and 8 demonstrate that the density and pH parameters are as expected, even after several heat shock cycles. Table 9 shows that there was a variation in viscosity from the second cycle of thermal shock. However, there was no significant change and the product remained fluid and with good flow. The presence of condensation water was also observed, which is expected because there is water in the composition. Other organoleptic parameters remained unchanged. Therefore, the sample containing the composition of lipid nanoparticles A is stable in view of test 3.

### Tests 4 and 5: Exposure to Different Temperatures and Sunlight

The stability of the composition of lipid nanoparticles A of the present invention was evaluated in view of different temperatures (Test 4) and exposure to sunlight (Test 5) in order to simulate other stress situations.

For this purpose, a sample was exposed to temperatures of 5° C., 25° C. and 40° C. for 90 days. At 0, 1, 7, 15, 30, 60 and 90 days, the organoleptic characteristics and physicochemical aspects of the sample were evaluated.

Additionally, the stability of the composition of lipid nanoparticles A was evaluated under sunlight. For this, the sample was exposed to sunlight for 90 days. At 0, 1, 7, 15, 30, 60 and 90 days, the organoleptic characteristics and physicochemical aspects of the sample were evaluated.

For Tests 4 and 5, the results refer to analysis of suspension separation, presence of oil on the surface, presence of condensation water, presence of clusters, presence of precipitate, color change, odor change, viscosity change, pH and density.

For Test 4, in all conditions, the formation of condensation water was observed, being an expected change due to the presence of water in the formula. When the sample was held at 40° C., a slight change in viscosity was observed—the product became less viscous. In 15 days at 40° C., a subtle phase separation occurred. However, when the sample was shaken, it became homogeneous again. After day 30 at 40° C., the sample showed a subtle color change, becoming lighter. After 60 days at 5° C., it was noticed that the sample became slightly more viscous. As the phase separation and changes in viscosity were not significant, the composition of lipid nanoparticles A was considered stable in view of Test 4.

For Test 5, both a small precipitate and a subtle color change occurred after 60 days of light exposure. The sample was considered stable in view of Test 5, and it is recommended not to expose the product to light.

### Completion of Tests

Tests 1 to 5 unequivocally indicated that the composition of lipid nanoparticles A is stable, presenting expected variations in pH and density under the conditions in which it was exposed. There was only a subtle color change in the sample exposed to light, suggesting that an embodiment of the present invention should be packaged in an opaque package and protected from light.

### Example 4—“Challenge Test”

Then, the sample of the composition of lipid nanoparticles A was studied by the challenge test, which aimed at evaluating the effectiveness of the preservative system (phenoxyethanol and caprylyl glycol, in the ratio of 1:1). The preservative system is necessary for satisfactory protection of the product against microbiological contamination, from manufacture to expiration date. The challenge consists of deliberately contaminating the sample with specific microorganisms and evaluating their growth at pre-defined time intervals up to 28 days. The microorganisms used in the challenge test were: Staphylococcus aureus (ATCC 6538), Pseudomonas aeruginosa (ATCC 9027), Escherichia coli (ATCC 8739), Aspergillus brasiliensis (ATCC 16404) and Candida albicans (ATCC 10231).

The test result is seen in Table 10.

TABLE 10
Evaluation of the effectiveness of the preservative system used
in the sample of the composition of lipid nanoparticles A.

| | | | Reinoculation | | |
|---|---|---|---|---|---|
| | Day 0 | Day 7 | (Day 7) | Day 14 | Day 28 |
| Escherichia coli | | | | | |
| Reading in CFU/mL: | 2.9 × 10³ | 0.0 | 0.0 | 0.0 | 0.0 |
| Reading in Log₁₀: | 3.46 | — | — | — | — |
| Staphylococcus aureus | | | | | |
| Reading in CFU/mL: | 1.5 × 10³ | 0.0 | 0.0 | 0.0 | 0.0 |
| Reading in Log₁₀: | 3.17 | — | — | — | — |
| Pseudomonas aeruginosa | | | | | |
| Reading in CFU/mL | 0.0 | 0.0 | 0.0 | 0.0 | 0.0 |
| Reading in Log₁₀: | — | — | — | — | — |
| Candida albicans | | | | | |
| Reading in CFU/mL: | 2.3 × 10⁵ | — | — | 0.0 | 0.0 |
| Reading in Log₁₀: | 5.36 | — | — | — | — |
| Aspergillus brasiliensis | | | | | |
| Reading in CFU/mL | 4.0 × 10³ | — | — | 0.0 | 0.0 |
| Reading in Log₁₀: | 3.60 | — | — | — | — |

After completion of the test, it was observed that the preservative system analyzed (phenoxyethanol and caprylyl glycol, in the ratio of 1:1) has antimicrobial efficacy, as it meets the study specifications.

### Example 5—Composition of Lipid Nanoparticles B (Comparative)

TABLE 11
Composition of lipid nanoparticles B
Composition

| Ingredient | Concentration (% w/w) |
|---|---|
| Water | 50-100% |
| Oleic acid | 5-10 |
| Capric/Caprylic Acid | 5-10% |
| Triglyceride | |
| Propanediol | 1-5% |
| Tocopheryl acetate | 1-10% |
| Glyceryl caprylate | 1-10% |
| Linoleic acid | 1-5% |
| Vitis vinifera bark Extract | 1.0% |
| Decyl glycoside | 5-10% |
| Lauryl glycoside | 5-10% |
| Bentonite | 1-5% |
| Citric acid | 0.1-1% |
| Cetearyl olivate | 1-10% |
| Sorbitan olivate | 1-10% |
| Glycerol undecylenate | 0.1-1% |

### Example 6—Composition of Lipid Nanoparticles C (Comparative)

TABLE 12
Composition of lipid nanoparticles C
Composition

| Ingredient | Concentration (% w/w) |
|---|---|
| Water | 50-100% |
| Oleic acid | 5-10% |
| Capric/Caprylic Acid | 5-10% |
| Triglyceride | |
| Glyceryl caprylate | 1-10% |
| Linoleic acid | 1-5% |
| Vitis vinifera bark Extract | 1.0% |
| Decyl glycoside | 5-10% |
| Lauryl glycoside | 5-10 |
| Bentonite | 1-5% |
| Citric acid | 0.1-1% |
| Cetearyl olivate | 1-10% |
| Sorbitan olivate | 1-10% |
| Glycerol undecylenate | 0.1-1% |

### Example 7—Composition of Lipid Nanoparticles D (Comparative)

TABLE 13
Composition of lipid nanoparticles D
Composition

| Ingredient | Concentration (% w/w) |
|---|---|
| Reverse osmosis water | 78% |
| Medium chain triglycerides | 7.0% |
| Cetyl palmitate | 5.0% |
| Polysorbate 80 | 3.5% |
| Ethoxydiglycol | 1.5% |
| Sorbitan oleate | 1.5% |
| Vitis vinifera extract | 1% |
| Sorbitan stearate | 1.0% |
| Oily vitamin E | 1.0% |
| Imidazolidinyl Urea | 0.5% |

### Example 8—Comparison of Stability of the Nanoparticle Compositions a to D

TABLE 14
Comparison of stability of compositions A, A′, A″, B, C and D.

| | Stability Test | |
|---|---|---|
| Composition | Result | Justification |
| A | Approved | Compatible and stable system. |
| | | Efficacy to prevent microbiological |
| | | contamination. |
| A′ | Failed | Encapsulating system incompatibility. |
| A″ | Failed | Encapsulating system incompatibility. |
| B | Failed | Encapsulating system incompatibility. |
| | | Ineffectiveness to avoid microbiological |
| | | contamination. |
| C | Failed | Encapsulating system incompatibility. |
| | | Ineffectiveness to avoid microbiological |
| | | contamination |
| D | Failed | Encapsulating system incompatibility. |
| | | Ineffectiveness to avoid microbiological |
| | | contamination |

### Example 9—Stability of Nanoparticle Compositions E and F, E′ and F′ (Comparative)

The compositions of lipid nanoparticles E and F, E′ and F′, were all prepared with 1% by weight of Vitis extract in relation to the total weight of the composition and have similar ingredients in their compositions. The compositions were tested for stability for a period of 30 days, kept at room temperature (without stress conditions).

Compositions E and F are produced without a preservative system. Qualitative and quantitative details of these comparative compositions are given in Table 15.

TABLE 15
Composition of lipid nanoparticles E and
F (without the use of preservative).

| | Composition E | Composition F |
|---|---|---|
| Ingredient | (% w/w) | (% w/w) |
| Vitis extract | 1 | 1 |
| Ethoxydiglycol | 1.5 | 1.5 |
| Cetyl Palmitate | 2.5 | 5 |
| Polysorbate 80 | 3 | 3.5 |
| Sorbitan Stearate | 1 | 1.5 |
| Medium chain | 7.5 | 7 |
| triglycerides | | |
| Oily Vitamin E | 1 | 1 |
| Reverse Osmosis Water | Qs. 100 | Qs. 100 |

Compositions E′ and F′ are produced with a preservative system. Qualitative and quantitative details of these comparative compositions are presented in Table 16.

TABLE 16
Composition of lipid nanoparticles E′ and
F′ (with use of preservative).

| | Composition E′ | Composition F′ |
|---|---|---|
| Ingredient | (% p/p) | (% p/p) |
| Vitis extract | 1 | 1 |
| Ethoxydiglycol | 1.5 | 1.5 |
| Cetyl Palmitate | 2.5 | 5 |
| Polysorbate 80 | 3 | 3.5 |
| Sorbitan Stearate | 1 | 1.5 |
| Medium chain | 7.5 | 7 |
| triglycerides | | |
| Oily Vitamin E | 1 | 1 |
| Hydroxyacetophenone | 0.5 | 0.5 |
| 1,2-Hexanediol 0.25% + | 0.5 | 0.5 |
| Caprylyl Glycol 0.25% ** | | |
| Reverse Osmosis Water | Qs. 100 | Qs. 100 |

* marketed as SymSave H ®.
** marketed as Sym Diol 68 ®.

After 30 days at room temperature (without stress conditions), compositions E′ and F′, with hydroxyacetophenone and 1,2-hexanediol 0.25%+caprylyl glycol 0.25% as preservatives, had changes in their organoleptic characteristics, with emphasis on the change in color to brown. Therefore, compositions E′ and F′ were discarded, showing that the choice of an inappropriate preservative system negatively impacts the stability of the composition.

After 30 days at room temperature (without stress conditions), compositions E and F were evaluated. Composition E remained purple in color. However, it showed a (non-homogeneous) phase separation and, therefore, was discarded. Composition F showed positive results of the 30-day stability test (without stress conditions).

Thus, composition F was selected for a test at a temperature of 45° C. In this test, the composition showed brown coloration and (non-homogeneous) phase separation.

The base of compositions E, F, E′ or F′ was also tested for compositions with concentrations of 2.5%, 5% and 10% of Vitis extract in relation to the total weight of the composition. On the other hand, even before the tests, the compositions showed unsatisfactory results ([FIG. 2]), given the formation of a paste with clusters.

The conclusion of this example is that only composition F is stable and homogeneous without being subjected to stress conditions, within quality parameters, containing 1% Vitis extract. However, under stress conditions (45° C.) this composition did not show stability. To verify the impact of the preservative system on the stability of composition F, other molecules with antimicrobial activity other than hydroxyacetophenone and 1,2-hexanediol 0.25%+caprylyl glycol 0.25% (composition F′) were tested, generating compositions G, H, I and J.

### Example 10—Stability of the Composition of Lipid Nanoparticles F in Different Preservative Systems

Keeping the base of the composition of lipid nanoparticles F described in example 9, different preservative systems were tested, generating the compositions G, H, I and J. The results are described in Table 17.

TABLE 17
Compositions G-J, with their respective stability results.

| Composition | Preservative | Observation |
|---|---|---|
| G | Pentylene glycol 3.2% | Great phase separation |
| H | Sodium benzoate 0.3% + | brown coloring |
| | imidazolidinyl urea 0.4% | |
| I | Cosmocil 0.5 | Three-phase separation |
| | | after centrifugation |
| J | Imidazolidinyl urea 0.5% | Selected Composition |

Composition J, which is composition F plus imidazolidinyl urea 0.5% preservative was stable in a preliminary stability test. This composition is shown in Table 18.

TABLE 18
Composition of nanoparticles J.

| | Composition J |
|---|---|
| Ingredient | (% w/w) |
| Vitis extract | 1 |
| Ethoxydiglycol | 1.5 |
| Cetyl Palmitate | 5 |
| Polysorbate 80 | 3.5 |
| Sorbitan Stearate | 1 |
| Sorbitan Oleate | 1.5 |
| Medium chain triglycerides | 7 |
| Oily Vitamin E | 1 |
| Imidazolidinyl Urea | 0.5 |
| Reverse Osmosis Water | Qs. 100 |

Composition J was then tested at room temperature and in an oven. The composition was considered stable in 90 days of study for both conditions tested. The average particle size was 186.6 nm. The formulation did not show instability trends and its encapsulation efficiency was 99.81%.

Composition J submitted to Challenge test also passed.

Composition J is also an embodiment of the present invention, as it has the ability to remain stable under test conditions. This composition is presented as a comparative example, because when compared to composition A, it presented long-term stability (greater than 90 days), but lower than the stability of composition A.

### Example 11—Encapsulation Effectiveness Study

The composition according to the present invention was subjected to centrifugation at 6000 rpm for 3 hours at room temperature using a microtube with filter (Ultrafree-MC Durapore Membrane PDVF 0.1 μm). The filtrate was collected and analyzed by the UV/Vis spectrophotometry technique.

15 mL of purified water, 1 mL of Folin-Ciocalteu reagent and 1 mL of the standard solution or 1 mL of the sample to be quantified were added to a test tube. Then, 3 ml of sodium carbonate 12.5% were added. The solutions were homogenized and placed in a water bath at 55° C. for 15 minutes. After this time, the samples were cooled and their absorbance was determined at a wavelength of 755 nm.

From the concentration of the standard solutions and the final volume of the analyzed solutions, the concentration of gallic acid for each point of the calibration curve was recalculated, in increasing order 0.00025 mg/mL; 0.0005 mg/ml; 0.0025 mg/ml; 0.005 mg/ml; 0.0075 mg/ml and 0.0125 mg/ml.

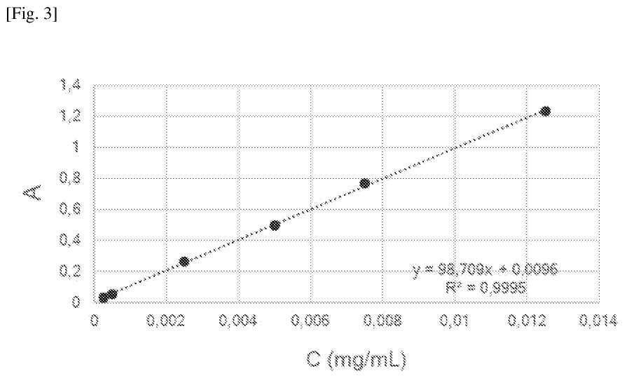

After reading the absorbance for each concentration, a calibration curve was constructed for gallic acid where the coefficient of determination (r²) was 0.9995. The curve and the equation obtained are shown in the graph of [FIG. 3].

The solution containing the sample had its absorbance value read and the concentration of polyphenols expressed as gallic acid was calculated using the equation of the line.

After processing the data, the concentration of total polyphenols that was not encapsulated, expressed as gallic acid, was found to be 0.002603 mg/mL. The composition tested had 1% of the active dry grape skin extract, the concentration of total polyphenols in the dry grape skin extract is 99.8%, according to the analysis performed.

In this sense, the composition according to the present invention has a concentration of 9.98 mg/ml of total polyphenols. The encapsulation efficiency was 99.97%.

### Example 9—Pre-Clinical Evaluation of Skin Permeation and Antioxidant Effect of Products Evaluated in an Ex Rivo Human Skin Experimental Model

Skin Permeation: xenobiotics, including drugs, cosmetic ingredients, agrochemicals and other products, can be absorbed through human skin and thus come into contact with skin tissue layers or even become systemically available. The consequences of this event include beneficial effects from drugs and cosmetic ingredients applied topically to the skin, as well as adverse effects from compounds to which the skin is exposed (e.g., occupational exposure). Thus, determining the absorption of individual compounds through human skin is of utmost importance for correct predictions of benefits and risks.

Assessment of antioxidant activity: a very common event after exposure to UV radiation is the formation of free radicals such as superoxides and hydroxyl radicals through the process of oxidative phosphorylation, causing damage to lipid and protein cellular constituents and especially to nucleic acids. The balance between the production of FRs and the natural antioxidant defense is essential for the maintenance of physiological homeostasis. If the FRs overload the body's ability to neutralize them, a condition known as oxidative stress sets in and increases the susceptibility of the skin tissue, impetuously evidencing the aesthetic changes resulting from the formation of radical proteins.

The understanding of the balance relationship between the production and neutralization of free radicals favored the development of research concerning oxidative damage markers using chemical or biological systems to evaluate the effectiveness of antioxidant substances. Among the models used, it can be cited the measurement of the fluorescence intensity emitted by the oxidation of the Dichloro-dihydro-fluorescein diacetate probe (DCFH-DA) in 2′-7′ dichlorofluorescein (DCF).

This technique allows us to evaluate the antioxidant effects of formulations against the extrinsic agents to which our skin is constantly exposed. Therefore, the early neutralization and/or inhibition of FRs can prevent the signaling cascade or chemical reactions that inevitably accelerate aging.

The analyzed samples were:
NVAC VITIS VINI (nanoparticle of the present invention in a concentration of 10%).

$$\begin{matrix}NVAC VITIS VINI 10 \% + FLUORESCEIN \\ NVAC VITIS VINI ( BLANK ) + FLUORESCEIN \\ EXTRACT SOLUTION 0.1 \% + FLUORESCEIN\end{matrix}$$

Human skin fragments obtained from elective plastic surgery were treated for 8 hours with the evaluated products NVAC VITIS VINI 10%, NVAC VITIS VINI 10%+FLUORESCEIN, NVAC VITIS VINI (BLANK)+FLUORESCEIN and EXTRACT SOLUTION 0.1%+FLUORESCEIN for subsequent assessment of skin permeation. In addition, the fragments were treated for 48 hours with the evaluated products and the market comparison product (benchmark): VIT C 10% MOISTURIZING CREAM for further exposure to ultraviolet radiation and evaluation of free radical synthesis using a fluorescent probe DCFH-DA (Dichloro-dihydro-fluorescein diacetate).

The skin fragments used in this study came from three (03) healthy individuals, female, phototype III, 29, 32 and 38 years old, who underwent elective plastic surgery in the abdomen region (abdominoplasty). After the surgical procedure, the skin fragments were collected in plastic bottles containing 0.9% saline solution and kept under refrigeration for up to 24 hours. This project did not include the storage of biological material for future use, and the spare fragments were properly disposed of as infectious waste. The use of human skin fragments from elective surgeries to carry out this study was submitted to the Research Ethics Committee of Universidade São Francisco—SP, CAAE 82685618.9.0000.5514, under opinion 2,493,285 (Annex III).

The skin fragments were fractionated into pieces of approximately 1.5 cm², placed in culture plates (Corning, USA) with DMEM culture medium containing 10% fetal bovine serum (FBS) and 0.1% gentamicin and kept in incubator at 37° C. in the presence of 5% CO₂.

To assess skin permeation, the skin fragments were treated with 15 mg of the evaluated products NVAC VITIS VINI 10%, NVAC VITIS VINI 10%+FLUORESCEIN, NVAC VITIS VINI (BLANK)+FLUORESCEIN and EXTRACT SOLUTION 0.1%+FLUORESCEIN for 8 hours. Then, the fragments were collected, submitted to histological sections for further analysis under fluorescent microscopy (OLYMPUS, JAP—BX53) using CellSens software (©2010 OLYMPUS CORPORATION). The fluorescence intensity parameter emitted by fluorescein was evaluated. After obtaining the images, the fluorescence intensity was quantified by using the ImageJ software (version 1.48, Arbitrary Units—A.U.).

For comparative evaluation of the antioxidant activity, the skin fragments were treated with 15 mg of the evaluated products and the market comparison product (benchmark): VIT C 10% MOISTURIZING CREAM. After 48 hours of incubation, the skin specimens were subjected to a dose of 10 J/cm² of UV, using the UVA Cube 400, SOL 500 H1 filter and UV Meter (Honle UV America Inc., MA, USA) devices. The cultures were incubated for an additional 24 hours with the evaluated products and the fragments were collected for labeling and semi-quantification of free radical synthesis, using the DCFH-DA probe.

After the treatment period, the skin fragments were embedded in Tissue-Tek® O.C.T.™ and then serial 12 micrometer histological sections were collected directly onto cryostat silanized slides (Leica, GER—CRYOCUT 1800).

Subsequently, the sections were washed with phosphate buffer (PBS) and incubated for 1 minute with a solution (1:10,000 in phosphate buffer) of Dichlorodihydro-fluorescein diacetate (DCFH-DA; Sigma, USA).

Immediately after this incubation, the slides were mounted using specific mounting media and analyzed under a microscope (OLYMPUS, JAP—BX53) using CellSens software (©2010 OLYMPUS CORPORATION). The fluorescence intensity parameter emitted by the oxidation of the DCFH-DA probe was evaluated. After obtaining the images, the fluorescence intensity was quantified using the ImageJ software (version 1.48, Arbitrary Units—A.U.).

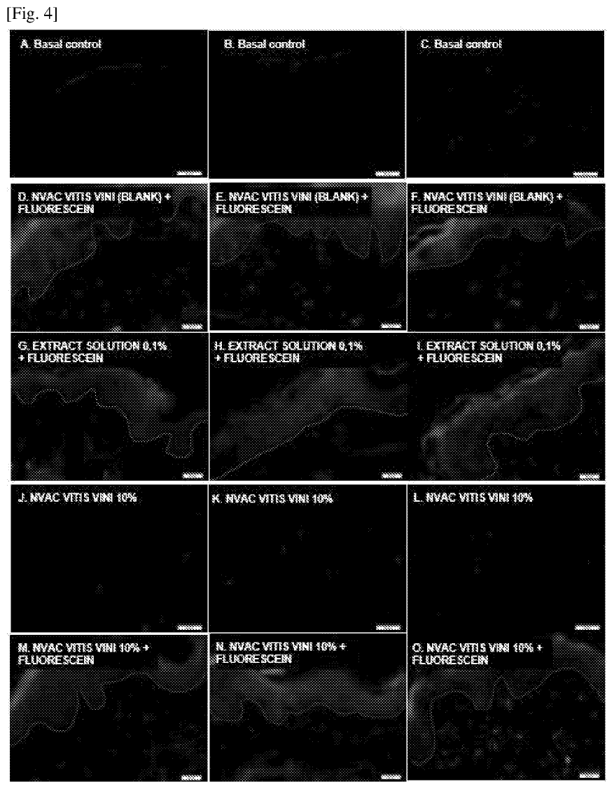

To assess skin permeation and semi-quantification of free radical synthesis using DCFH-DA, the ANOVA test was used, which also allowed the measurement of the results variation, by comparing data between groups. Then, the Bonferroni post-test was applied, which reinforced and made the result presented in the ANOVA test even more accurate. A significance level of 5% was used in both assessments (GraphPad Prism v6).

[FIG. 4] represents the results obtained from skin permeation of the evaluated products NVAC VITIS VINI (BLANK)+FLUORESCEIN; EXTRACT SOLUTION 0.1%+FLUORESCEIN; NVAC VITIS VINI 10% and NVAC VITIS VINI 10%+FLUORESCEIN, in human skin culture. As we can see, the positive test control—NVAC VITIS VINI (BLANK)+FLUORESCEIN, as well as the evaluated products SOLUTION EXTRACT 0.1%+FLUORESCEIN and NVAC VITIS VINI 10%+FLUORESCEIN showed an increase in fluorescence intensity when compared to the baseline control. As expected, the evaluated product NVAC VITIS VINI 10% showed low fluorescence intensity, similar to the baseline control.

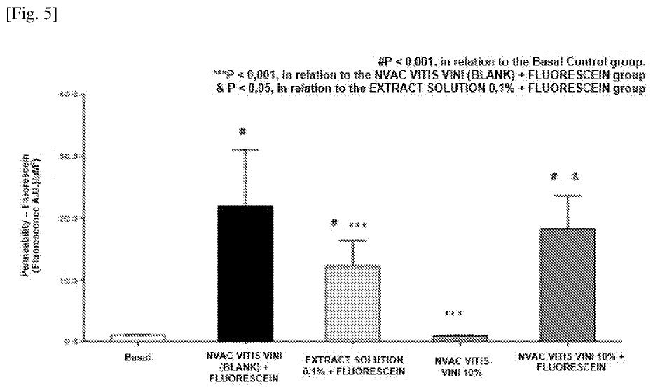

In a complementary way, [FIG. 5] presents the semi-quantification results of the fluorescence obtained from the analysis of microscopic images. As we can see, the treatment with the evaluated products NVAC VITIS VINI (BLANK)+FLUORESCEIN; EXTRACT SOLUTION 0.1%+FLUORESCEIN and NVAC VITIS VINI 10%+FLUORESCEIN promoted increases of 21.19; 11.79 and 17.70 fold, respectively, of the fluorescence intensities (dermis+epidermis), when compared to the baseline control (P<0.001). It was also observed that the fluorescence intensities observed for the fragments treated with SOLUTION EXTRACT 0.1%+FLUORESCEIN and NVAC VITIS VINI 10%+FLUORESCEIN were considered statistically different in relation to those treated with the evaluated product NVAC VITIS VINI (BLANK)+FLUORESCEIN (P<0.001).

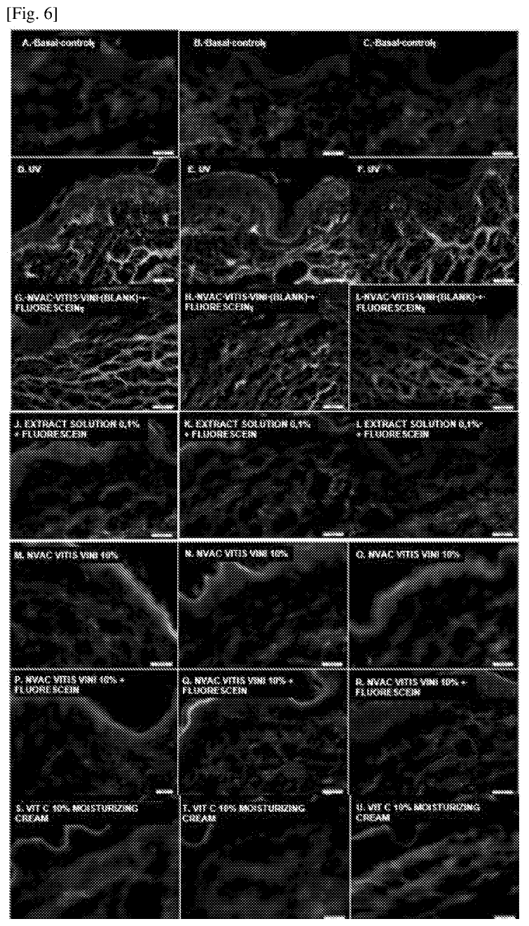

In addition, the skin fragments treated with the evaluated product NVAC VITIS VINI 10%+FLUORESCEIN showed higher fluorescence intensity than the fragments treated with EXTRACT SOLUTION 0.1%+FLUORESCEIN (P<0.05), mainly in the viable epidermis regions and dermis ([FIG. 1]—G-I and M-O). In this sense, the results suggest that the encapsulated product showed more significant skin penetration when compared to the product in its free form.

[FIG. 6] represents the antioxidant effects of the evaluated products NVAC VITIS VINI (BLANK)+FLUORESCEIN, EXTRACT SOLUTION 0.1%+FLUORESCEIN, NVAC VITIS VINI 10% and NVAC VITIS VINI 10%+FLUORESCEIN, in human skin culture submitted to UV radiation. As expected, radiation exposure produced an increase of 187.60% (P<0.001) in the FRs labeling/production when compared to the unexposed control, thus contributing to the process of oxidative stress installation.

The evaluated product NVAC VITIS VINI (BLANK)+FLUORESCEIN, as expected, did not demonstrate a protective effect in preventing the production of FRs, when compared to the UV group. On the other hand, the products evaluated SOLUTION EXTRACT 0.1%+FLUORESCEIN, NVAC VITIS VINI 10% and NVAC VITIS VINI 10%+FLUORESCEIN demonstrated a protective effect, preventing the production of FRs.

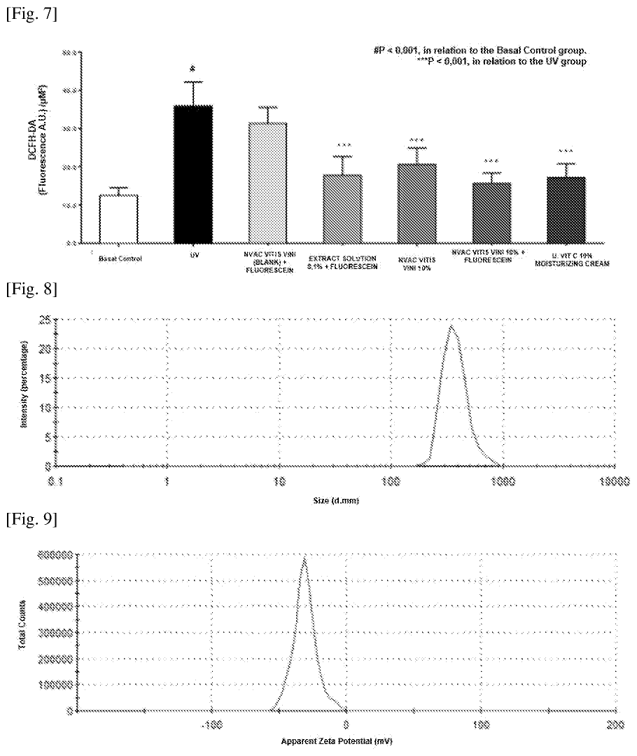

Complementarily, in [FIG. 7], we present the results of the semi-quantification of the FRs labeling/production obtained from the analysis of microscopic images. As we can see, the treatment with the evaluated products EXTRACT SOLUTION 0.1%+FLUORESCEIN, NVAC VITIS VINI 10% and NVAC VITIS VINI 10%+FLUORESCEIN promoted a reduction of 50.47%; 42.39% and 56.30%, respectively, in the production of FRs, when compared to the UV group (P<0.001).

In the same way, the market comparison product VIT C 10% MOISTURIZING CREAM promoted a decrease of 52.27% in the synthesis of FRs, also when compared to the UV group (P<0.001).

The skin permeation results showed that the evaluated product NVAC VITIS VINI 10%+FLUORESCEIN showed improved skin penetration when compared to the product EXTRACT SOLUTION 0.1%+FLUORESCEIN. In addition, products evaluated EXTRACT SOLUTION 0.1%+FLUORESCEIN, NVAC VITIS VINI 10% and NVAC VITIS VINI 10%+FLUORESCEIN promoted a reduction in the oxidative stress of the skin, exerting a protective effect against the excessive increase in the synthesis of free radicals, induced by exposure to UV radiation. In the same way, the market comparison product VIT C 10% MOISTURIZING CREAM promoted a decrease in the production of free radicals also after exposure to UV radiation. Thus, these results reveal that the evaluated products exerted an antioxidant and anti-aging effect, protecting the skin from the deleterious effects of exposure to solar radiation.

### Example 10—Characterization—Result Using the Nanozetasizer for Evaluating the Size

In the analysis using nanozetasizer for size evaluation, it was observed that the particles have an average diameter of 362 nm, with a monodisperse size distribution and an average surface charge of −30.9 mV. The results are considered within the scope of the present invention. The size and surface charge can be seen in FIGS. 8 and 9, respectively.

### Example 11—Gene Modulation Assay

The effect of gene modulation, within the scope of the present invention, was determined by using a panel containing several genes. Upregulation and downregulation effects were observed at multiple UV irradiations (UVA-6 J/cm² (+UVB −310 mJ/cm²)×4 days) on the gene expression profile in a full-thickness reconstructed skin model.

Downregulation was observed in genes responsible for extracellular matrix synthesis (COL1A1, COL3A1, ELN, FN1, FBN1), cellular and oxidative stress response (SEMA3A), DNA repair (PCNA), differentiation of keratinocytes (CALML5, CASP14, FLG, KRT10, LOR, KRT1), hydration (AQP3), intercellular junctions (DSC1, DSG1) and desquamation (KLK7).

Upregulation was observed in genes responsible for growth factors (NGF, EGF, FGF2), extracellular matrix degradation (MMP1, MMP3, PTGS2, TFPI2), response to cellular and oxidative stress/inflammation (FOXO3, HMOX1, MT1E, MT1H, TXNRD1), DNA repair (XPA, ERCC3), differentiation of keratinocytes (IVL, TGM1, TCHH), innate immunity (DEFB4A), proliferation of keratinocytes (KRT19), inflammation (IL1B, IL8).

The composition of the present invention was able to modulate genes related to skin aging.

Those skilled in the art will make use of the knowledge presented herein, which makes it possible to reproduce the invention in the embodiments presented and in other variants, covered by the scope of the appended claims.

1. A composition of lipid nanoparticles containing Vitis vinifera extract, comprising from 0.1 to 2.0% by weight of Vitis vinifera extract in relation to the total weight of the composition, wherein the physical delimitation of the nanoparticles is a lipid matrix.

2. The composition according to claim 1, wherein the composition comprises from 0.5 to 1.5% by weight of Vitis vinifera extract in relation to the total weight of the composition.

3. The composition according to claim 2, wherein the composition comprises 1.0% by weight of Vitis vinifera extract in relation to the total weight of the composition.

4. The composition according to claim 1, wherein the Vitis vinifera extract comprises gallic acid, catechin, epicatechin, procyanidin B1 and procyanidin B2.

5. The composition according to claim 1, wherein the composition comprises 0.1 to 3% by weight of preservatives.

6. The composition according to claim 5, wherein the preservative is selected from the group consisting of: phenoxyethanol, caprylyl glycol, BHT, disodium EDTA, sodium metabisulfite, parabens, Lonicera japonica, Lonicera caprifolium, hydroxyacetophenone, 1,2-hexanediol, 1,2-octanediol, tropolone, pentylene glycol, sodium benzoate, potassium sorbate, iodopropynyl-butylcarbamate, imidazolidinyl urea, polyaminopropyl biguanide, and mixtures thereof.

7. The composition according to claim 6, wherein the composition comprises 0.1 to 2% phenoxyethanol and 0.1 to 2% caprylyl glycol.

8. The composition according to claim 7, wherein the preservative comprises a 1:1 combination of phenoxyethanol and caprylyl glycol.

9. The composition according to claim 1, wherein the composition comprises 10 to 40% w/w of lipids.

10. The composition according to claim 9, wherein the composition comprises from 10 to 40% by weight of lipids in relation to the total weight of the composition, and wherein the lipid is selected from the group consisting of: simple or complex fatty acids, long, medium or short chain triglycerides, polypolylene glycol stearyl ester, and mixtures thereof.

11. The composition according to claim 10, wherein the composition comprises 5% to 10% capric/caprylic acid triglyceride, 5% to 10% oleic acid, 1% to 10% linoleic acid, and 1% to 5% PPG-15 stearyl ether.

12. The composition according to claim 1, wherein the composition comprises 1 to 10% surfactants selected from the group consisting of ethoxylated nonionic surfactants, poloxamer, and mixtures thereof.

13. The composition according to claim 12, wherein the composition comprises 1 to 5% steareth-2 (stearyl alcohol ethoxylate), 1 to 5% poloxamer 407 (polymer with oxirane) and 0.1 to 2% steareth-21 (stearyl alcohol ethoxylate).

14. The composition according to claim 1, wherein the composition has a soft color from soft purple to pink, wherein the soft color is defined as a color mixed with white.

15. The composition according to 1, wherein the particles have an average diameter of 300 to 420 nm.

16. The composition according to claim 15, wherein the particles have an average diameter of 360 nm.

17. The composition according to claim 1, wherein the particles have an average surface charge of −10 to −50 mV.

18. The composition according to claim 17, wherein that the particles have an average surface charge of −30 mV.

19. A method for preventing skin aging, with antioxidant, anti-aging, anti-inflammatory, whitening, photoprotective, skin permeation and gene modulation related to skin aging effects, comprising: applying a cosmetic product comprising the composition as defined in claim 1 to skin.

20. The method according to claim 19, wherein the composition downregulates genes responsible for extracellular matrix synthesis, cellular and oxidative stress response, DNA repair, differentiation of keratinocytes, hydration, intercellular junctions and desquamation, and upregulates genes responsible for growth factors, extracellular matrix degradation, cellular and oxidative stress response/inflammation, DNA repair, differentiation of keratinocytes, innate immunity, proliferation of keratinocytes, and inflammation.

21. An antioxidant dermocosmetic product for preventing aging of skin, comprising at least one composition of lipid nanoparticles as defined in claim 1, and cosmetically suitable excipients.

22. The product according to claim 21, wherein the product is liquid, semi-solid, or solid.

23. The product according to claim 22, wherein the product is a lotion or a serum.

24. The product according to claim 23, wherein the product is an oil-in-water or water-in-oil emulsion.

25. The product according to claim 24, wherein the product is used in the preparation of a cream or a gel-cream.

26. The product according to claim 25, wherein the product is used in the preparation of a gel.

27. A skin care method comprising applying at least one layer of a product as defined in claim 21 to skin.

28. The method according to claim 20, wherein the downregulated genes responsible for extracellular matrix synthesis comprise COL1A1, COL3A1, ELN, FN1, and FBN1, the downregulated genes responsible for cellular and oxidative stress response comprise SEMA3A, the downregulated genes responsible for DNA repair comprise PCNA, the downregulated genes responsible for differentiation of keratinocytes comprise CALML5, CASP14, FLG, KRT10, LOR, and KRT1, the downregulated genes responsible for hydration comprise AQP3, the downregulated genes responsible for intercellular junctions comprise DSC1 and DSG1, the downregulated genes responsible for desquamation comprise KLK7, the upregulated genes responsible for growth factors comprise NGF, EGF, and FGF2, the upregulated genes responsible for extracellular matrix degradation comprise MMP1, MMP3, PTGS2, and TFPI2, the upregulated genes responsible for cellular and oxidative stress response/inflammation comprise FOXO3, HMOX1, MT1E, MT1H, and TXNRD1, the upregulated genes responsible for DNA repair comprise XPA and ERCC3, the upregulated genes responsible for differentiation of keratinocytes comprise IVL, TGM1, and TCHH, the upregulated genes responsible for innate immunity comprise DEFB4A, the upregulated genes responsible for proliferation of keratinocytes comprise KRT19, and the upregulated genes responsible for inflammation comprise IL1B and IL8.
